SEQUENCE LISTING (51 sequences)

SEQ ID NO: 1 - DNA, 24 bp, Artificial sequence
  Common sequence
atgagaattctagtagggatgtag

SEQ ID NO: 2 - DNA, 32 bp, Artificial sequence
  Common Sequence
atgttaaaaagttgaaaggttaatgtaaaaca

SEQ ID NO: 3 - DNA, 19 bp, Artificial sequence
  Common Sequence
actggttggttgtgtggaa

SEQ ID NO: 4 - DNA, 35 bp, Artificial sequence
  Common Sequence
gatctgtctgatcgtttctttagtggtgtcattta

SEQ ID NO: 5 - DNA, 25 bp, Artificial sequence
  Common Sequence
aatgaggccagttatcttctataac

SEQ ID NO: 6 - DNA, 18 bp, Artificial sequence
  SMN1-F Primer
atgttaaaaagttgaaag

SEQ ID NO: 7 - DNA, 19 bp, Artificial sequence
  SMN1-R Primer
gagaattctagtagggatg

SEQ ID NO: 8 - DNA, 14 bp, Artificial sequence
  SMN2-F Primer
tggttggttgtgtg

SEQ ID NO: 9 - DNA, 23 bp, Artificial sequence
  SMN2-R Primer
gatcgtttctttagtggtgtcat

SEQ ID NO: 10 - DNA, 28 bp, Artificial sequence
  SMN1+6 Primer
cattcctttagtttccttacagggtatc

SEQ ID NO: 11 - DNA, 24 bp, Artificial sequence
  SMN2+6 Primer
ccttaattttccttacagggattt

SEQ ID NO: 12 - DNA, 24 bp, Artificial sequence
  SMN1+100 Primer
ttacattaacctttcaactattta

SEQ ID NO: 13 - DNA, 22 bp, Artificial sequence
  SMN2+100 Primer
acattaacctttcaacattcta

SEQ ID NO: 14 - DNA, 28 bp, Artificial sequence
  SMN1+6 Primer
cattcctttagtttccttacagggtatc

SEQ ID NO: 15 - DNA, 24 bp, Artificial sequence
  SMN2+6 Primer
ccttaattttccttacagggattt

SEQ ID NO: 16 - DNA, 23 bp, Artificial sequence
  SMN1+215 Primer
gtgaaagtatgtttcttccagat

SEQ ID NO: 17 - DNA, 21 bp, Artificial sequence
  SMN2+215 Primer
gaaagtatgtttcttcctcac

SEQ ID NO: 18 - DNA, 22 bp, Artificial sequence
  Amel-F Primer
ccctgggctctgtaaagaatag

SEQ ID NO: 19 - DNA, 24 bp, Artificial sequence
  Amel-R Primer
atcagagcttaaactgggaagctg

SEQ ID NO: 20 - DNA, 22 bp, Artificial sequence
  D5S818-F Primer
ctcccatctggatagtggacct

SEQ ID NO: 21 - DNA, 23 bp, Artificial sequence
  D5S818-R Primer
atagcaagtatgtgacaagggtg

SEQ ID NO: 22 - DNA, 21 bp, Artificial sequence
  Th01-F Primer
aggctctagcagcagctcatg

SEQ ID NO: 23 - DNA, 23 bp, Artificial sequence
  Th01-R Primer
gaaaagctcccgattatccagcc

SEQ ID NO: 24 - DNA, 21 bp, Artificial sequence
  E1-F Primer
ggagggcgataaccactcgta

SEQ ID NO: 25 - DNA, 21 bp, Artificial sequence
  E1-R Primer
ccacaactccagtgagcggat

SEQ ID NO: 26 - DNA, 22 bp, Artificial sequence
  E2a-F Primer
tcttaccctttccagagcgatg

SEQ ID NO: 27 - DNA, 23 bp, Artificial sequence
  E2a-R Primer
aggctatcaacttctaaaggagg

SEQ ID NO: 28 - DNA, 22 bp, Artificial sequence
  E2b-F Primer
cggagccttgagactagcttat

SEQ ID NO: 29 - DNA, 24 bp, Artificial sequence
  E2b-R Primer
ggactaatgagacatcctttgaag

SEQ ID NO: 30 - DNA, 22 bp, Artificial sequence
  E3-F Primer
acctccccactgatcaaaacga

SEQ ID NO: 31 - DNA, 23 bp, Artificial sequence
  E3-R Primer
ctcatctagtctctgcttccaga

SEQ ID NO: 32 - DNA, 22 bp, Artificial sequence
  E4-F Primer
ccaccgaggcattaattttttc

SEQ ID NO: 33 - DNA, 25 bp, Artificial sequence
  E4-R Primer
actttcatagaaggtttacctttcc

SEQ ID NO: 34 - DNA, 24 bp, Artificial sequence
  E5-F Primer
gtataaaacaaatattctgggtaa

SEQ ID NO: 35 - DNA, 25 bp, Artificial sequence
  E5-R Primer
gggatgttctacaatgacattttac

SEQ ID NO: 36 - DNA, 26 bp, Artificial sequence
  E6-F Primer
atctttttctgtctccagataattcc

SEQ ID NO: 37 - DNA, 25 bp, Artificial sequence
  E6-R Primer
tgcaagagtaatttaagcctcagac

SEQ ID NO: 38 - DNA, 22 bp, Artificial sequence
  E8-F Primer
gcatagagcagcactaaatgac

SEQ ID NO: 39 - DNA, 29 bp, Artificial sequence
  E8-R Primer
ccaataattatccaatatcattcaaaatc

SEQ ID NO: 40 - DNA, 19 bp, Artificial sequence
  5C>G Primer
cccgcgggtttgctatcgg

SEQ ID NO: 41 - DNA, 27 bp, Artificial sequence
  305G>A Primer
tcaaggggacaaatgttctgccatcta

SEQ ID NO: 42 - DNA, 29 bp, Artificial sequence
  400G>A Primer
gtacgtttacactggatatggaaatcgaa

SEQ ID NO: 43 - DNA, 19 bp, Artificial sequence
  683T>A Primer
caccaccaccaccccacga

SEQ ID NO: 44 - DNA, 21 bp, Artificial sequence
  689C>T Primer
accaccaccccacttacaatt

SEQ ID NO: 45 - DNA, 21 bp, Artificial sequence
  785G>T Primer
tgatgctgatgctttggcaat

SEQ ID NO: 46 - DNA, 23 bp, Artificial sequence
  815A>G Primer
tgttcatggtacatgagtggcgg

SEQ ID NO: 47 - DNA, 22 bp, Artificial sequence
  821C>T Primer
ggtacatgagtggctatcatgt

SEQ ID NO: 48 - DNA, 24 bp, Artificial sequence
  830A>G Primer
gagtggctatcatactggctagtg

SEQ ID NO: 49 - DNA, 27 bp, Artificial sequence
  835-1G>A Primer
gattaacttcctttattttccttacaa

SEQ ID NO: 50 - DNA, 23 bp, Artificial sequence
  863G>T Primer
agacaaaatcaaaaagaagcaat

SEQ ID NO: 51 - DNA, 20 bp, Artificial sequence
  g.27134T>G Primer
taacatctgaactttttaac

(19) United States
(12) Patent Application Publication
(10) Pub. No.: US 2022/0170102 A1
(43) Pub. Date: Jun. 2, 2022

(54) METHOD FOR DETECTING SMN GENE COPY NUMBER USING SMNP AS REFERENCE

(71) Applicant: BEIJING MICROREAD GENETICS CO., LTD, Beijing (CN)

(72) Inventors: Ye ZHANG, Beijing (CN); Yihui WANG, Beijing (CN); Qi ZHANG, Beijing (CN); Yan MENG, Beijing (CN); Haiyan ZHU, Beijing (CN); Yuying LIU, Beijing (CN); Chuguang CHEN, Beijing (CN)

(73) Assignee: BEIJING MICROREAD GENETICS CO., LTD, Beijing (CN)

(21) Appl. No.: 17/616,996

(22) PCT Filed: Jun. 1, 2020
(86) PCT No.: PCT/CN2020/093694

§ 371 Date: Dec. 6, 2021

(30) Foreign Application Priority Data
Jun. 6, 2019 (CN) 2019 10492012.3

(51) Int. Cl. C12Q 1/6883; C12Q 1/6858; C12Q 1/6853

(52) U.S. Cl. CPC C12Q 1/6883; C12Q 1/6858; C12Q 2600/156; C12Q 2600/118; C12Q 1/6853

(57) ABSTRACT

Provided in the present invention is a method for detecting copy numbers of survival of motor neuron genes SMN1 and/or SMN2 in a target genome, the method comprising: amplifying target regions in the SMN1 and/or SMN2 genes and an SMNP gene in a genome by using specific primer combinations, and then using an SMNP amplified product as a reference to determine the copy numbers of the SMN1 and/or SMN2 genes by means of comparing relative amounts of the amplified product. Further provided in the present invention is a reagent kit for detecting the copy numbers of SMN1 and/or SMN2.

### CROSS REFERENCE TO RELATED APPLICATIONS

This application claims the priority of a Chinese patent application filed with the Chinese Patent Office with an application number of 201910492012.3 on Jun. 6, 2019, and the entire content of which is incorporated into this application by reference.

### FIELD

The present disclosure relates to the technical field of biotechnology, specifically to a method and a detection kit for detecting the copy number of survival motor neuron (SMN) genes in vitro.

### BACKGROUND

Spinal muscular atrophy (SMA) is a relatively common autosomal recessive genetic disease. The carrier rate and incidence of SMA are basically the same among all races, the carrier rate is 1/40 to 1/60, and the incidence in newborns is about 4-10/100,000.

SMA is a neuromuscular disorder characterized by degeneration of motor neuron cells in the anterior horn of the spinal cord, which can cause symmetrical and progressive muscle weakness and muscle atrophy in the limbs and trunk (Neurotherapeutics. 2008; 5: 499-506).

According to the patient's age of onset, athletic ability, and life span, the International SMA consortium meeting divides typical SMA into three types: type I, type II, and type III (Neuromuscular Disorders. 1991; 1(2): 81; Neuromuscular disorders. 1992; 2(5-6):423-428).

SMA type I, onset 6 months ago, is also called Werdnig-Hoffmann disease, infantile SMA and so on. This type accounts for about 50% of all SMA patients. Children with this type of SMA have severe progressive muscle weakness and weakened muscle tone, and generally cannot sit alone. The survival period will not exceed 2 years if there is no treatment.

SMA type II, onset at 6 to 18 months, is also called juvenile SMA, intermediate SMA and so on. Some can sit and some can stand, but most can't walk, and often suffer from respiratory dysfunction. The general life expectancy is more than two years, and some can survive to adolescence or even longer.

SMA type III, onset after 18 months, is also called Kugelberg-Welander disease, mild SMA and so on. It has strong clinical heterogeneity and usually develops, but the proximal muscles of infancy usually have problems. Most patients are able to walk with only slight weakness. Adolescence may recur and the life expectancy is normal.

In addition to the typical SMA types I, II and III, there are also types 0 and IV.

SMA type 0, congenital type, has severe joint contractures, facial paralysis and respiratory failure, and often has spontaneous fractures and is extremely weak. The disease began before birth and died within one month after birth.

SMA type IV, adult type, usually onset after 35 years of age, mainly manifested by slow and gradual proximal weakness and muscle atrophy of the upper and lower limbs, and can walk in adulthood.

The most important pathogenic gene of SMA is located in the 5q13.2 region of chromosome 5. This region as a whole presents a huge inverted repeat structure, which also causes non-allelic homologous recombination in this region, causing abnormalities such as gene deletion or duplication.

The two highly homologous genes in this region are named survival motor neuron (SMN). Studies have found that SMN1, which is close to the telomere, is the pathogenic gene of SMA. SMA will be caused if both SMN1 gene copies are missing or have pathogenic mutations; SMN2 near the centrosome is not a pathogenic gene of SMA, but its copy number is related to the severity of SMA clinical manifestations (Cell 80:155-165, 1995).

The SMN gene is 20 kb in full length and contains 9 exons (1, 2a, 2b, 3-8). The SMN1 and SMN2 gene sequences are highly consistent in all gene regions including the promoter. It has been found that SMN1 and SMN2 genes have only 5 different bases (r51454173648, INS6-45, G/A; rs4916, Exon 7+6, 840th base, C/T; rs212214, INS7+100A/G; rs1244569826, INS7+215, A/G; rs1323191655, Exon8+245G/A), they are distributed in the region between intron 6 and exon 8. All exon sequences of SMN1 and SMN2 genes differ by only two bases. One is a different base in exon 7, which is a synonymous mutation; the other is a different base in exon 8. After the stop codon, it has no effect on protein coding. Therefore, the amino acid sequences encoded by SMN1 and SMN2 are completely identical.

The SMN gene transcription product is about 1.7 kb in length and encodes a 294 amino acid SMN protein, which is involved in the formation of a multi-protein complex related to RNA processing. SMN protein is universally expressed in human tissues, and motor neurons in the anterior horn of the spinal cord have a high demand for them. If the expression level of SMN protein is too low, neurons will die and cause muscle atrophy.

SMN1 can express stable and fully functional SMN protein. Although SMN2 and SMN1 gene sequences are very similar, most of the transcripts transcribed by SMN2 in vivo are not spliced correctly, and only about 10% of mRNAs are spliced correctly and translated into SMN protein with normal activity. Most transcripts lack the seventh exon and are called Δ7SMN2. These proteins lack normal SMN protein function and are quickly degraded. Therefore, SMN2 cannot fully compensate for the lack of SMN protein caused by SMN1 gene deletion or mutation, but its copy number will affect the severity of the disease in patients with SMN1 deletion SMA.

The reason for the different splicing of SMN1 and SMN2 is that the two genes have one different base on exon 7. The sixth position of exon 7 (Exon 7+6) is the 840th nucleotide in the coding region. It is C in SMN1 and T in SMN2 (840C>T). This base difference is believed to affect the structure and function of the splicing enhancer in this region, and results in difference of RNA splicing. Therefore, the base at this position, 840C>T, is the key base that affects whether a normal SMN protein can be produced, and it is also the key base that distinguishes the functional difference between SMN1 and SMN2.

Due to the high consistent of SMN1 and SMN2 sequences, and their inverted repeat structure on the chromosome, non-allelic homologous recombination may occur between the two genes. This recombination is an important reason for the deletion or duplication of the SMN gene, as well as the conversion of SMN1 and SMN2 genes.

In the human genome, SMNP (survival of motor neuron 1, telomeric pseudogene) has the highest homology with the exon regions of SMN1 and SMN2. SMNP is located in the 9p21.3 region, with a total length of about 1643 bp. All 9 exons and the last intron of SMN1 have corresponding homologous fragments in SMNP, and the homologous sequences between SMN1 mRNA and SMNP are higher than 80%. It is speculated that SMNP is a pseudogene formed by reverse transcription of SMN RNA. There are no other genes within 1 MB upstream and downstream of SMNP. The region where SMNP is located has no inverted repeat structure similar to the genomic region where SMN1 and SMN2 are located, so non-allelic homologous recombination and copy number changes will not occur. At present, there is no clear function report of SMNP gene, and it is not related to SMA disease.

95˜98% of SMA patients have a homozygous deletion of SMN1 exon 7 and/or exon 8 (Hum. Genet. 12:1015-1023, 2004), and about 5% of patients have a SMN1 heterozygous deletion and the only remaining copies of SMN1 have pathogenic mutations or other unknown causes (Am J. Hum. Genet. 64: 1340-1356, 1999). Therefore, quantitative detection of copy number of SMN1 gene exon 7 is the main strategy for SMA gene screening and prenatal molecular diagnosis.

The detection of SMN1 copy number can effectively screen SMA carriers. However, there is no effective detection method for the “2+0” type among SMA carriers. “2+0” means that the individual has 2 copies of SMN1, but the two copies are on one chromosome and there is no SMN1 copy on the other chromosome. This genotype is also a carrier of SMA, and it is possible to pass chromosomes without SMN1 to offspring, resulting in SMA carriers or even patients. There are reports that some SNP sites are linked to “2+0” in Jews, and have a certain correlation with “2+0” in some other races (Human Mutation; 2000, 15: 228).

The particularity and difficulty of SMN1 copy number detection are:

1. It must be able to effectively distinguish 0 copy (patient), 1 copy (carrier) and 2 or more copies (normal people). If it can only distinguish between presence and absence, it can only be used for patient diagnosis, not for carrier screening. For carrier screening, which demands effectively distinguish 1 from 2 or more copies, accurate and stable quantitative capabilities are especially required.

2. SMN1 and SMN2 genes are highly homologous, and they differ only by 5 bases. The detection needs to have good specificity, so that the detection signal of SMN1 is not affected by SMN2.

Currently, the common methods for detecting SMN1 copy number are as follows:

1. MLPA (Multiplex Ligation-Dependent Probe Amplification)

This method uses multiple sets of specific probes to hybridize with SMN1 exon 7 and other related positions as well as a large number of control sites, followed by ligation and amplification. The SMN1 copy number are quantitatively determined by comparing the product amounts of SMN1 exon 7 and control sites. As there has been no more accurate method for SMN1 copy number detection for a long time, the MLPA method is the most widely used method in scientific research and clinical detection. However, this method has complicated operation, high cost, high requirements for the samples to be tested, and complicated data analysis. Moreover, each test requires multiple control samples to be tested at the same time, and the test results shall be corrected based on the results of the control samples. Improper selection of the control samples or abnormal results will also lead to detection errors.

2. qPCR

qPCR has excellent quantification ability on a larger scale, but it is not good at distinguish between one copy or two copies of a gene. Theoretically, it is only 1 CT value difference between the CT value of 1 copy and the CT value of 2 copies. It requires high level for detection stability and repeatability for effectively distinguish the samples with CT difference of 1. In addition, qPCR uses relative quantification, and other control gene signals and SMN1 signals need to be used to calculate ACT. Ensuring that the primers and probes corresponding to SMN1 and control genes maintain the same amplification efficiency is the premise of effective detection. If the detection conditions change (including amplification conditions, system composition and volume, sample concentration and purity, etc.), the amplification efficiency maintained by different primers and probes will be different, which will affect the detection results. In a word, qPCR is a feasible method, but it has higher requirements for actual quality and detection condition control.

3. ddPCR (Droplet Digital PCR)

This method has good quantitative ability and can effectively check the copy number of SMN1. In addition to complex operations and high costs, ddPCR also requires the detection of internal reference genes, similar to qPCR, which requires high detection stability and repeatability.

4. High resolution melting curve (HRM)

This method uses a pair of common primers to amplify the relevant regions of SMN1 and SMN2 exon 7 and determine the melting curve of the product. Due to the individual base differences in the SMN1 and SMN2 sequences, the melting curve peaks of the two homozygous double-strands and hybrid double-strands are different, which will cause the two products to show specific patterns on the melting curves. The problem with this method is that the copy number ratio of SMN1 and SMN2 can be quantitatively determined, but the final value cannot be determined. For example, it is impossible to distinguish SMN1:SMN2=1:2 or 2:4, and it is impossible to detect samples with SMN2 copy number of 0. In order to solve these problems, other tests need to be introduced, such as determining the total copy number of SMN1 and SMN2. Since SMN1 and SMN2 have more copy number combinations, it is inconvenient and error-prone to distinguish different melting curve styles.

5. NGS (Next Generation Sequencing)

The usual method is to amplify or capture the relevant regions of SMN1 and SMN2 exon 7, a library was constructed and sequenced. And the copy number ratio of SMN1 and SMN2 is determined according to the ratio of 840C/T. But only this result cannot determine the final value of the copy number, and other methods are needed to obtain the total copy number of SMN1 and SMN2. The calculation of the total copy number can be determined according to a specific algorithm based on the number of reads of the sequencing results of a large number of other genes detected at the same time and the total number of reads of the SMN gene. The above method can effectively detect SMN1 copy number, but the operation is complicated, the cost is high, and the result calculation is complicated, and a large number of internal reference gene detection is also required, which requires high control of reaction conditions.

In addition, Sanger sequencing, single-strand conformation polymorphism analysis (PCR-SSCP), denaturing high performance liquid chromatography (DHPLC) and other methods are also available in the art. These methods have low quantitative ability, poor stability, cumbersome operation, and may need other tests to correct the results. They are not suitable for large-scale clinical testing. Therefore, there is an urgent need for a rapid and accurate detection method to quantify the copy number of SMN1 and/or SMN2 genes.

### SUMMARY

In view of the existing problems and deficiencies of the current SMN1 and/or SMN2 genes copy number quantitative detection methods, an object of the present disclosure is to provide a method for accurately detecting the copy number of SMN1 and/or SMN2 genes by using SMNP as a control.

The inventors used the endogenous homologous pseudogene SMNP as the reference gene, and after extensive analysis and experiments, the inventors designed primers that can simultaneously amplify SMN1 and SMNP, or simultaneously amplify SMN2 and SMNP, or simultaneously amplify SMN1, SMN2 and SMNP, and realized the simultaneous amplification and detection of the target region and the control region with the same primers and the same binding ability. In this way, the differences from different primers caused by various conditions can be avoided. Regardless of how the reaction conditions, concentration of system components, inhibitors and other factors affect the amplification efficiency, their impact on the target region and the control region is the same, that is, the amplification results tolerate various conditions and adverse factors, so that improving the quantitative ability and detection stability.

In order to detect the copy number of SMN, firstly it is necessary to distinguish between SMN1 and SMN2 genes. The two differ only by 5 bases in the entire gene region, and they can only be distinguished by these 5 bases. To achieve this objective, the present disclosure provides the following technical solutions.

The inventors compared the homology between SMN1 and SMNP sequences, and the results are shown in FIG. 1. According to the results, among the 5 bases where SMN1 and SMN2 are inconsistent, the sequence near INS6-45, Exon7+6 and the upstream sequence have poor homology with SMNP sequence; the sequence near Exon8+245 also has poor homology with SMNP sequence; the sequence near INS7+100 and INS7+215 and the upstream sequence have good homology with the SMNP sequence.

Among the 5 difference sites/positions, the first choice for detection is Exon7+6 (840C>T) position located on the 7th exon, which is a functional site that leads to different splicing modes of SMN1 and SMN2. However, the sequences around and upstream of this position have poor homology with SMNP, so it is difficult to design primers to realize co-amplification of SMN1 or SMN2 and SMNP. Therefore, the inventors designed detection primers based on regions with good sequence homology, especially those near INS7+100 and INS7+215.

Using the methods of the present disclosure, not only SMNP is amplified at the same time, but also SMN1 and SMN2 are distinguished. Specifically, at the INS7+100 site, the base of SMN1 and SMNP is A, while SMN2 is G. The primers at this site can amplify both SMN1 and SMNP, but not SMN2. Similarly, in INS7+215 site, the base of SMN2 and SMNP is G, while SMN1 is A. The primers at this site can simultaneously amplify SMN2 and SMNP, but not SMN1. The copy number of SMN1 or SMN2 can be determined by comparing the relative amounts of different products.

However, for the detection of INS7+100 and INS7+215, the detection result reflects the copy number of INS7+100 and INS7+215, not the copy number of Exon7+6. Although these positions are very close, there is no guarantee that they will be closely linked. In fact, there will be a certain rate of conversion between SMN1 and SMN2. If a conversion happens exactly between the detection site and Exon7+6 site, the detection result will be biased. In order to avoid the deviation of the detection result caused by the conversion, the inventor provides a method for detecting whether there is a conversion, and the first detection result can be corrected according to the conversion type. While performing conversion detection, primers can also be added to detect more relevant positions, such as other control sites, SMA-related pathogenic sites and “2+0” related sites.

The technical solution provided by the present disclosure uses a pair of primers to simultaneously amplify the sequence of SMN1/2 containing Exon7+6 sites, and determine the copy number ratio of the two. At the same time, the regions that can satisfy the simultaneous amplification of SMN1, SMN2 and SMNP with the same primers are used as control to determine the total copy number of SMN1 and SMN2. As the same primers and the same binding ability are used to simultaneously amplify and detect the corresponding regions of the three genes, the amplification test results can withstand various conditions and various interference factors, and the reads number of the corresponding products of each gene can reflect the copy number of the template more accurately. In this way, the total copy number of SMN1 and SMN2 can be effectively determined with only a small number of sites, without using a large number of control sites, which reduces the system complexity and cost, and does not need to use more complex algorithms to correct the results.

The present disclosure provides a method for detecting copy number of motor neuron survival genes SMN1 and/or SMN2 in a target genome, comprising amplifying a target region of SMN1 and/or SMN2 genes and SMNP gene in the target genome with a primer combination, and comparing amounts of amplified products of SMN1 and/or SMN2 genes by using SMNP amplified product as a reference; and determining the copy number of SMN1 and/or SMN2 genes.

The method provided by the present disclosure comprising the following steps:

1) providing a sample containing the target genomic DNA;

2) in the presence of the primer combination, using the genomic DNA of step 1) as a template and amplifying the target region of SMN1 and/or SMN2 genes and SMNP gene recognized by the primer combination; and

3) detecting the amplified products, and using the SMNP amplified product as the reference to determine the copy number of SMN1 and/or SMN2 genes in the target genome;

wherein,

the primer combination for SMN1 gene is primer combination 1, which amplifies the target regions of SMN1 and SMNP genes but not SMN2 gene in the genome, and the SMN1 amplified product and SMNP amplified product are distinguishable in the detection;

the primer combination for SMN2 gene is primer combination 2, which amplifies the target regions of SMN2 and SMNP genes but not SMN1 gene in the genome, and the SMN2 amplified product and SMNP amplified product are distinguishable in the detection; and/or

the primer combination for SMN1 and SMN2 genes is primer combination 3, which amplifies the target regions of SMN1, SMN2 and SMNP genes in the genome, and the SMNP amplified product is distinguishable from the amplified products of SMN1 and SMN2 in the detection.

In an embodiment of the present disclosure, in step 3) the length and amount of the amplified products are detected, and wherein for the primer combination 1, the lengths of the amplified products of SMN1 and SMNP are different; for the primer combination 2, the lengths of the amplified products of SMN2 and SMNP are different; and/or for the primer combination 3, the lengths of the amplified products of SMN1, SMN2 and SMNP are different.

Wherein, in step 3), the amplified product is detected by a method selected from the following: electrophoresis, fluorescence quantification, and mass spectrometry, such as capillary electrophoresis.

In an embodiment of the present disclosure, the method of the present disclosure further comprises detecting gene conversion between SMN1 and SMN2 genes. For example, detecting the conversion between INS7+100 and Exon7+6; and/or detecting the conversion between INS7+215 and Exon7+6.

In an embodiment of the present disclosure, the first primer of the primer combination 1 is located in a first consensus sequence region of SMN1 and SMNP genes, and the sequence of the first primer is identical or complementary to at least a part of the first consensus sequence, for example, the first consensus sequence is SEQ ID NO: 1 (ATGAGAATTCTAGTAGGGATGTAG), and the first primer sequence is preferably SEQ ID NO: 7 (GAGAATTCTAGTAGGGATG).

In an embodiment of the present disclosure, the second primer of the primer combination 1 is located in a second consensus sequence region of SMN1 and SMNP genes, the corresponding sequence of the SMN2 gene is not consistent with the second consensus sequence, and the sequence of the second primer is complementary or identical to at least a part of the second consensus sequence, for example, the second consensus sequence is SEQ ID NO: 2 (ATGTTAAAAAGTTGAAAGGTTAATGTAAAACA), and the second primer sequence is preferably SEQ ID NO: 6 (ATGTTAAAAAGTTGAAAG).

In an embodiment of the present disclosure, the third primer of the primer combination 2 is located in a third consensus sequence region of SMN2 and SMNP genes, the corresponding sequence of the SMN1 gene is not consistent with the third consensus sequence, and the sequence of the third primer is complementary or identical to at least a part of the third consensus sequence, for example, the third consensus sequence is SEQ ID NO: 3 (ACTGGTTGGTTGTGTGGAA), and the third primer sequence is preferably SEQ ID NO: 8 (TGGTTGGTTGTGTG).

In an embodiment of the present disclosure, the fourth primer sequence of the primer combination 2 is located in a fourth consensus sequence region of SMN2 and SMNP genes, and the sequence of the fourth primer is complementary or identical to at least a part of the fourth consensus sequence, for example, the fourth consensus sequence is SEQ ID NO: 4 (GATCTGTCTGATCGTTTCTTTAGTGGTGTCATTTA) or SEQ ID NO: 5 (AATGAGGCCAGTTATCTTCTATAAC). The fourth primer sequence is preferably SEQ ID NO: 9 (GATCGTTTCTTTAGTGGTGTCAT).

In the method provided by the present disclosure, at least one primer in the primer combination is modified or substituted with a modified base to replace a normal base. For example, the modification is selected from fluorescent modification, phosphorylation modification, phosphorothioate modification, locked nucleic acid modification and peptide nucleic acid modification; the primer sequence in the primer combination has one or more nucleotide substitutions, additions, or deletions compared with the complementary sequence of the corresponding region on the template, while retaining its ability to initiate amplification reaction.

The amplification in the method of the present disclosure is carried out by polymerase chain reaction (PCR), and the PCR amplification is carried out in one or more reaction systems.

The present disclosure also provides a method for diagnosing the risk or severity of spinal muscular atrophy (SMA) in a subject or its offspring, which includes detecting the copy number of motor neuron survival genes SMN1 and/or SMN2 in the genome of the subject.

The invention also provides a kit for detecting copy number of SMN1 and/or SMN2 genes.

### BRIEF DESCRIPTION OF DRAWINGS

FIG. 1: sequence homology comparison between SMN1 and SMNP. “*” indicates the bases consistent in SMN1 and SMNP sequences, the box indicates 5 bases that are not consistent in SMN1 and SMN2, and the underline indicates the sequences of SMN1 exon 7 and exon 8.

FIG. 2: using SMNP as a control, the copy number of SMN1 and SMN2 were determined by ARMS PCR. FIG. 2A shows the results of DNA sample from SMA patient, FIG. 2B shows the results of DNA sample from SMA carrier, and FIG. 2C shows the results of DNA sample from normal people.

FIG. 3: the detection result of whether there is a conversion between SMN1 and SMN2. FIG. 3A shows the detection result of a normal sample without conversion, and FIG. 3B shows the detection result of a sample with conversion.

FIG. 4: the results of detection of SMN gene copy number and SMA-related sites through two amplification reactions.

FIG. 5: scatter plot of peak area ratios obtained from 2802 samples.

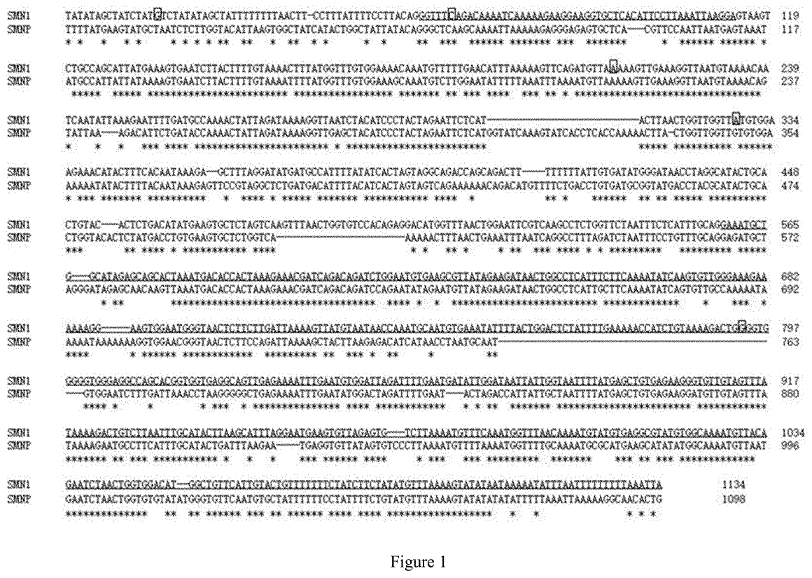

### DETAILED DESCRIPTION

### Example 1 Determination of Copy Number of SMN1 and SMN2 Using SMNP as a Control by ARMS (Amplification Refractory Mutation System) PCR

The primers used include:

SMN1-F:
(SEQ ID NO: 6)
5′AT(+G)TTAAAAAGTTGAAAG 3′;
SMN1-R:
(SEQ ID NO: 7)
5′FAM-GAGAATTCTAGTAGGGATG 3′;
SMN2-F:
(SEQ ID NO: 8)
5′TG(+G)TTGGTTGTGTG 3′;
SMN2-R:
(SEQ ID NO: 9)
5′FAM-GATCGTTTCTTTA(+G)TGGTGTCAT 3′.

“(+G)” means that the G base at this position is modified with LNA (Locked Nucleic Acid), which is used to enhance primer binding ability and improve primer specificity indirectly.

Wherein, SMN1-F is completely consistent with SMN1 and SMNP sequence, but not consistent with SMN2 sequence; SMN2-F is completely consistent with SMN2 and SMNP sequence, but not consistent with SMN1 sequence; SMN1-R and SMN2-R are consistent with SMN1, SMN2 and SMNP sequences completely.

The combination of SMN1-F and SMN1-R can specifically amplify SMN1 and SMNP, but not SMN2. The SMN1 and SMNP products amplified by the two primers are 103 bp and 100 bp, respectively. The combination of SMN2-F and SMN2-R can specifically amplify SMN2 and SMNP, but not SMN1. The SMN2 and SMNP products amplified by the two primers are 293 bp and 283 bp, respectively. The two pairs of primers can be used alone or together. Each product can be identified by detecting the size of the product.

In addition to primers, the PCR amplification system also contains the following components: DNA polymerase (2G Robust, KAPA Biosystems); UDPase enzyme; amplification buffer.

Three samples were tested, including one case of SMA patient (SMN1 copy number is 0), one case of carrier (SMN1 copy number is 1), and one case of normal person (SMN1 copy number is 2). The copy number of SMN1 and SMN2 genes in each sample have been determined by MLPA method.

The specific detection steps are as follows:

1) peripheral blood samples were collected and genomic DNA was extracted;

2) PCR amplification reaction system was prepared, each amplification system includes: 4 primers mixture 5 μl, amplification buffer 10 μl, DNA polymerase and UDPase enzyme 1 μl, DNA of the sample to be tested 1 μl, make up 200 μl with sterile water;

3) PCR amplification was performed, the reaction conditions were: 50° C., 5 minutes; 95° C., 5 minutes; 30 cycles of 94° C., 30 seconds, 58° C., 30 seconds, 72° C., 30 seconds; 72° C., 10 minutes;

4) the amplified product was subjected to capillary electrophoresis;

5) data analysis, related files were imported into GeneMapper software, including Panel, Bin, corresponding analysis method, internal standard file, sample source data (.fsa file), the previously imported file was selected in the relevant parameter selection column, and the data was analyzed.

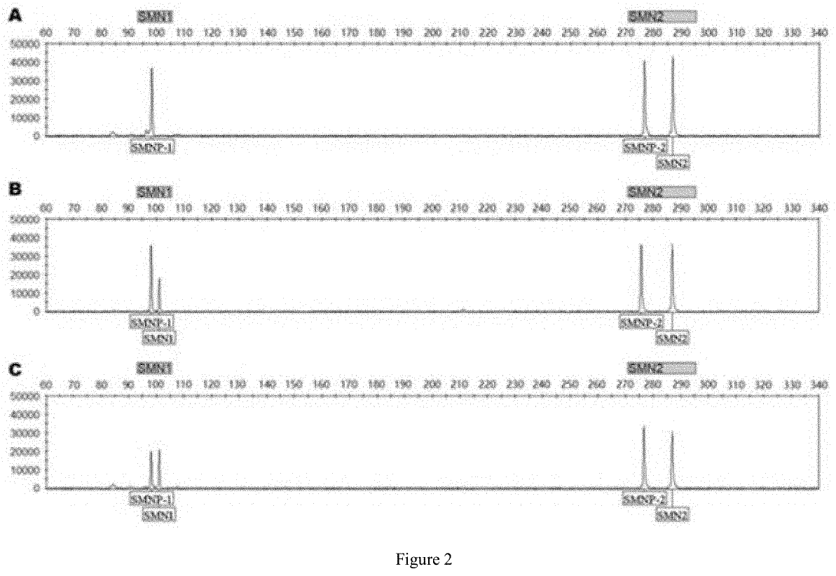

The results of capillary electrophoresis are shown in FIG. 2. FIG. 2A shows the results of DNA sample from SMA patient, FIG. 2B shows the results of DNA sample from SMA carrier, and FIG. 2C shows the results of DNA sample from normal people.

As shown in FIG. 2, there are several product peaks in the expected size range, and the corresponding template can be determined according to the product size. For the two products for detecting SMN1 copy number, the expected sizes of SMN1 and SMNP products are 103 bp and 100 bp, respectively. The peak area ratio of the two products reflects the ratio of the two products, which is the ratio of the corresponding initial template amount, that is, the ratio of SMN1 and SMNP copy numbers. Similarly, for the two products for detecting SMN2 copy numbers, the sizes of SMN2 and SMNP products are expected to be 293 bp and 283 bp, respectively. The ratio of the peak area of the two products reflects the ratio of the amount of the two products, which is the ratio of the corresponding initial template amount, that is, the ratio of SMN2 and SMNP copy numbers.

The peak area ratios of SMN1 to SMNP and SMN2 to SMNP of the three samples were 0.00, 1.07; 0.49, 1.05; 1.03, 0.95, respectively. Given that the SMNP copy number in the genome is 2, it can be concluded that the copy number of SMN1 and SMN2 in the three samples tested are: 0, 2; 1, 2; 2, 2, respectively. This result is completely consistent with the MLPA result. It shows that the method for detecting SMN1 and SMN2 gene copy numbers with SMNP as a control provided by the present disclosure is accurate and intuitive, and does not rely on other control sites, and does not require complex correction algorithms.

### Example 2 Detection of Conversion Between SMN1 and SMN2

Due to the high consistency of SMN1 and SMN2 sequence, and their inverted repetitive structure on the chromosome, non-allelic homologous recombination may occur between the two genes. This recombination is an important reason for the deletion or duplication of the SMN gene, as well as the conversion of the SMN1 and SMN2 genes. This conversion may cause the exon 7+6 position on exon 7 not to be linked with other different bases. Primers can be designed to conduct another PCR reaction to detect whether there is conversion. If there is conversion, the original detection results can be corrected according to the conversion type.

The following first set of primers is used to detect the conversion between INS7+100 and Exon7+6, including four primers:

SMN1 + 6:
(SEQ ID NO: 10)
5′CATTCCTTTAGTTTCCTTACAGGGTATC 3′;
SMN2 + 6:
(SEQ ID NO: 11)
5′CCTTAATTTTCCTTACAGGGATTT 3′;
SMN1 + 100:
(SEQ ID NO: 12)
5′HEX-TTACATTAACCTTTCAACTATTTA 3′;
SMN2 + 100:
(SEQ ID NO: 13)
5′HEX-ACATTAACCTTTCAACATTCTA 3′.

The second set of primers is used to detect the conversion between INS7+215 and Exon7+6, including four primers:

SMN1 + 6:
(SEQ ID NO: 14)
5′CATTCCTTTAGTTTCCTTACAGGGTATC 3′;
SMN2 + 6:
(SEQ ID NO: 15)
5′CCTTAATTTTCCTTACAGGGATTT 3′;
SMN1 + 215:
(SEQ ID NO: 16)
5′HEX-GTGAAAGTATGTTTCTTCCAGAT 3′;
SMN2 + 215:
(SEQ ID NO: 17)
5′HEX-GAAAGTATGTTTCTTCCTCAC 3′.

Among them, the sequence of SEQ ID NO: 10 is consistent with the sequence of SEQ ID NO: 14, and the sequence of SEQ ID NO: 11 is consistent with the sequence of SEQ ID NO: 15.

The underlined bases are artificially designed bases that are not consistent with the genome sequence, which is used to increase primer specificity and balance amplification efficiency.

The two sets of primers can be amplified separately or simultaneously in the same PCR reaction.

Among them, SMN1+6 can specifically bind to the template with Exon7+6 position as the C base, that is, the corresponding sequence of SMN1. SMN2+6 can specifically bind to the template with Exon7+6 position as the T base, that is, the corresponding sequence of SMN2. Similarly, SMN1+100 and SMN2+100 can specifically bind to the corresponding sequences of SMN1 and SMN2 at INS7+100, respectively, and SMN1+215 and SMN2+215 can specifically bind to the corresponding sequences of SMN1 and SMN2 at INS7+215, respectively.

Amplified products of the first set of primers have different sizes according to different types of templates, and can be used to determine whether and what kind of conversion occurs between INS7+100 and Exon7+6.

In normal SMN1 gene, Exon7+6 is C and INS7+100 is A. The primers SMN1+6 and SMN1+100 correspond to these two sites respectively, so that PCR amplification is realized and the size of the amplified product is 197 bp. In the normal SMN2 gene, Exon7+6 is T and INS7+100 is G. Primers SMN2+6 and SMN2+100 correspond to these two sites respectively, and the size of the amplified product is 191 bp. If conversion occurs, Exon7+6 is T and INS7+100 is A, then the primers SMN2+6 and SMN1+100 can correspond to these two sites respectively, and the size of the amplified product is 193 bp. In this situation, the SMN1 copy number calculated by the copy of INS7+100 will be higher than the copy number calculated by Exon7+6. This type of conversion is named type I conversion in the present disclosure. Exon7+6 of the SMN gene that undergoes conversion is T, which is the same as SMN2, and will show similar functions as SMN2 in vivo. On the other hand, if after conversion Exon7+6 is C and INS7+100 is G, then primers SMN1+6 and SMN2+100 can correspond to these two sites respectively, and the size of the amplified product is 195 bp. In this situation, the SMN1 copy number calculated by the copy number of INS7+100 will be lower than the copy number calculated by Exon7+6. This type of conversion is named type II conversion in the present disclosure. The converted SMN gene Exon7+6 is C, which is the same as SMN1, and it will show similar functions as SMN1 in vivo.

Similarly, the second set of primers can be used to determine whether and what kind of conversion occurs between INS7+215 and Exon7+6. The size of the amplified products of normal SMN1 gene, normal SMN2 gene, type I conversion and type II conversion between the two sites are 315 bp, 309 bp, 311 bp, and 313 bp, respectively.

In addition to primers, the PCR amplification system also contains the following components: DNA polymerase (2G Robust, KAPA Biosystems); UDPase enzyme; amplification buffer.

The tested samples are human peripheral blood samples, and the copy number of SMN1 and SMN2 genes in each sample have been tested by the MLPA method.

The specific detection steps are as follows:

1) peripheral blood samples were collected;

2) PCR amplification reaction system was prepared, each amplification system includes: 5 μl of a mixture with a total of 6 primers, 10 μl amplification buffer, 1 μl DNA polymerase and UDPase enzyme, 1 μl blood sample to be tested, and make up 20 μl with sterile water;

3) PCR amplification was performed, the reaction conditions are: 50° C., 5 minutes; 95° C., 5 minutes; 30 cycles of 94° C., 30 seconds, 58° C., 30 seconds, 72° C., 30 seconds; 72° C., 10 minutes;

4) the amplified products were subjected to capillary electrophoresis;

5) data analysis was performed, related files was imported into GeneMapper software, including Panel, Bin, corresponding Analysis Method, internal standard file, sample source data (.fsa file), the previously imported file was selected in the relevant parameter selection column, and the data was analyzed.

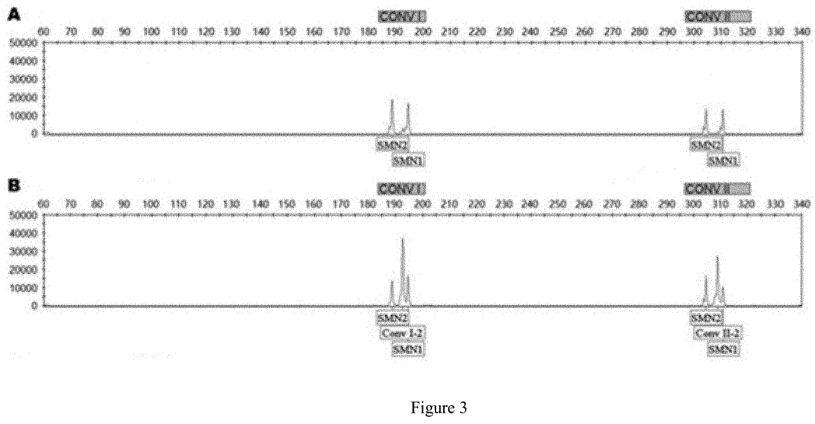

The results of electrophoresis are shown in FIG. 3.

Among the test results shown in FIG. 3A, in the range corresponding to conversion between INS7+100 and Exon7+6 (panel “CONN I”), there are only products corresponding to SMN1 and SMN2, and no product corresponding to conversion; in the range corresponding to conversion between INS7+215 and Exon7+6 (panel “CONN II”), there are only products corresponding to SMN1 and SMN2, and no product corresponding to conversion. This result indicates that there is no conversion in this sample.

Among the test results shown in FIG. 3B, in the detection area corresponding to conversion between INS7+100 and Exon7+6, in addition to the products corresponding to SMN1 and SMN2, there are also products corresponding to type II conversion; in the detection area corresponding to conversion between INS7+215 and Exon7+6, in addition to the products corresponding to SMN1 and SMN2, there are also products corresponding to type II conversion. This result indicates that the sample has a conversion, occurs between INS7+100 and Exon7+6, which is a type II conversion.

The result obtained by the method in Example 1 is actually the copy number of SMN1 at INS7+100 and the copy number of SMN2 at INS7+215. And what needs to be detected is actually the copy number of Exon7+6 site. In most of the samples, there was no conversion of the SMN gene between INS7+100 and Exon7+6, and between INS7+215 and Exon7+6. The detected result is the copy number of SMN1 and SMN2 at Exon7+6. However, if conversion occurs, the copy number obtained by the method of Scheme 1 is not consistent with the copy number of Exon7+6 site and needs to be corrected.

For example, FIG. 3B in this embodiment corresponds to a sample, and the result of the sample detected by the method in Example 1 is 1 copy for SMN1 gene and 2 copies for SMN2 gene. The detection result of the detection system of this example shows that the sample has type II conversion at the corresponding position. In other words, there are Exon7+6 site as C (SMN1 type), INS7+100 site as T (SMN2 type), and Exon7+6 site as C (SMN1 type), and INS7+215 site as T (SMN2 type). At this time, calculating SMN1 copy number by base copy number at INS7+100 will be lower than that calculated by Exon7+6, and calculating SMN2 copy number by base copy number at INS7+215 will be higher than Exon7+6.

Combined with the conversion result, the copy number of SMN1 gene in this sample is 2 (or more), and the copy number of SMN2 gene is 1 (the result of the detection conversion system has SMN2 products, so it will not be less than 1).

If no correction is made, the copy number of SMN1 in this sample is 1, and it will be misjudged as a carrier of SMA. After correction, the SMN copy number of this sample is 2 (or more), which is normal.

Validated by the MLPA method, the copy number of exon 7 of the SMN1 gene in this sample is 2, and the copy number of exon 8 of this sample is 1. The copy number of exon 7 of SMN2 gene is 1, and the copy number of exon 8 is 2. This result is consistent with our result. Moreover, it can be seen from the MLPA results that the SMN gene of the sample has indeed undergone conversion, which causes inconsistent copy number of exons 7 and 8.

### Example 3 Simultaneous Detection of SMN1 and SMN2 Copy Numbers and Various SMA Related Sites

As mentioned above, the SMN gene copy number is detected through two PCR amplification reactions. One amplification is used to detect the copy number of SMN1 and SMN2, and the other amplification is used to detect whether there is a conversion between SMN1 and SMN2. In addition, primers for other sites have been added to the system, including SMA-related pathogenic SNPs, “2+0” related sites, and other control sites.

The primers of the first reaction include:

| | | | Theoretical |
|---|---|---|---|
| | | | length |
| SEQ | | | of |
| ID | | | product |
| NO: | Primer | Sequence | (bp) |
| 6 | SMN1-F | AT(+G)TTAAAA | 100/103 |
| | | AGTTGAAAG | |
| 7 | SMN1-R | FAM-GAGAATTCT | |
| | | AGTAGGGATG | |
| 8 | SMN2-F | TG(+G)TTGGTTG | 283/293 |
| | | TGTG | |
| 9 | SMN2-R | FAM-GATCGTTTC | |
| | | TTTA(+G)TGGTG | |
| | | TCAT | |
| 18 | Amel-F | HEX-CCCTGGGCT | 106/112 |
| | | CTGTAAAGAATAG | |
| 19 | Amel-R | ATCAGAGCTTAAA | |
| | | CTGGGAAGCTG | |
| 20 | D5S818-F | HEX-CTCCCATCT | 264-300 |
| | | GGATAGTGGACCT | |
| 21 | D5S818-R | ATAGCAAGTATGT | |
| | | GACAAGGGTG | |
| 22 | Th01-F | HEX-AGGCTCTAG | 216-254 |
| | | CAGCAGCTCATG | |
| 23 | Th01-R | GAAAAGCTCCCGA | |
| | | TTATCCAGCC | |

The primers of the second reaction include:

| | | | Theoretical |
|---|---|---|---|
| | | | length |
| SEQ | | | of |
| ID | | | product |
| NO: | Primer | Sequence | (bp) |
| 10 | SMN1 + 6 | CATTCCTTTAGTTTCCTTACAGGGTATC | 191/193/ |
| 11 | SMN2 + 6 | CCTTAATTTTCCTTACAGGGATTT | 195/197 |
| 12 | SMN1 + 100 | HEX-TTACATTAACCTTTCAACTATTTA | |
| 13 | SMN2 + 100 | HEX-ACATTAACCTTTCAACATTCTA | |
| 24 | E1-F | GGAGGGCGATAACCACTCGTA | 350 |
| 25 | E1-R | FAM-CCACAACTCCAGTGAGCGGAT | |
| 26 | E2a-F | TCTTACCCTTTCCAGAGCGATG | 260 |
| 27 | E2a-R | FAM-AGGCTATCAACTTCTAAAGGAGG | |
| 28 | E2b-F | CGGAGCCTTGAGACTAGCTTAT | 270 |
| 29 | E2b-R | FAM-GGACTAATGAGACATCCTTTGAAG | |
| 30 | E3-F | ACCTCCCCACTGATCAAAACGA | 339 |
| 31 | E3-R | FAM-CTCATCTAGTCTCTGCTTCCAGA | |
| 32 | E4-F | CCACCGAGGCATTAATTTTTTC | 242 |
| 33 | E4-R | FAM-ACTTTCATAGAAGGTTTACCTTTCC | |
| 34 | E5-F | GTATAAAACAAATATTCTGGGTAA | 303 |
| 35 | E5-R | FAM-GGGATGTTCTACAATGACATTTTAC | |
| 36 | E6-F | ATCTTTTTCTGTCTCCAGATAATTCC | 283 |
| 37 | E6-R | FAM-TGCAAGAGTAATTTAAGCCTCAGAC | |
| 38 | E8-F | GCATAGAGCAGCACTAAATGAC | 315 |
| 39 | E8-R | FAM-CCAATAATTATCCAATATCATTCAAAATC | |
| 40 | 5C > G | CCCGCGGGTTTGCTATCGG | 212 |
| 41 | 305G > A | TCAAGGGGACAAATGTTCTGCCATCTA | 249 |
| 42 | 400G > A | GTACGTTTACACTGGATATGGAAATCGAA | 156 |
| 43 | 683T > A | CACCACCACCACCCCACGA | 152 |
| 44 | 6890T | ACCACCACCCCACTTACAATT | 150 |
| 45 | 785G > T | TGATGCTGATGCTTTGGCAAT | 224 |
| 46 | 815A > G | TGTTCATGGTACATGAGTGGCGG | 196 |
| 47 | 8210T | GGTACATGAGTGGCTATCATGT | 189 |
| 48 | 830A > G | GAGTGGCTATCATACTGGCTAGTG | 182 |
| 49 | 835-1G > A | GATTAACTTCCTTTATTTTCCTTACAA | 202 |
| 50 | 863G > T | AGACAAAATCAAAAAGAAGCAAT | 169 |
| 51 | g.27134T > G | TAACATCTGAACTTTTTAAC | 175 |
| 40 | 5C > G | CCCGCGGGTTTGCTATCGG | 212 |
| 41 | 305G > A | TCAAGGGGACAAATGTTCTGCCATCTA | 249 |
| 42 | 400G > A | GTACGTTTACACTGGATATGGAAATCGAA | 156 |
| 43 | 683T > A | CACCACCACCACCCCACGA | 152 |
| 44 | 6890T | ACCACCACCCCACTTACAATT | 150 |
| 45 | 785G > T | TGATGCTGATGCTTTGGCAAT | 224 |
| 46 | 815A > G | TGTTCATGGTACATGAGTGGCGG | 196 |
| 47 | 8210T | GGTACATGAGTGGCTATCATGT | 189 |
| 48 | 830A > G | GAGTGGCTATCATACTGGCTAGTG | 182 |
| 49 | 835-1G > A | GATTAACTTCCTTTATTTTCCTTACAA | 202 |
| 50 | 863G > T | AGACAAAATCAAAAAGAAGCAAT | 169 |
| 51 | g.27134T > G | TAACATCTGAACTTTTTAAC | 175 |
| 18 | Amel-F | HEX-CCCTGGGCTCTGTAAAGAATAG | 106/112 |
| 19 | Amel-R | ATCAGAGCTTAAACTGGGAAGCTG | |
| 20 | D5S818-F | HEX-CTCCCATCTGGATAGTGGACCT | 264-300 |
| 21 | D5S818-R | ATAGCAAGTATGTGACAAGGGTG | |
| 22 | Th01-F | HEX-AGGCTCTAGCAGCAGCTCATG | 216-254 |
| 23 | Th01-R | GAAAAGCTCCCGATTATCCAGCC | |

The first reaction system is used to detect SMN1 and SMN2 copy numbers. In addition to the four primers used in Example 1 for determining the copy number, three pairs of primers were also set to amplify the sex chromosome locus Amel, and two STR sites D5S818 and TH01. In addition to serve as routine controls, these three pairs of primers can also monitor whether samples are contaminated and prevent the mix up of samples.

The second reaction system is used to detect whether a conversion occurs between SMN1 and SMN2. In addition to the four primers used to detect the conversion between INS7+100 and Exon7+6 in Example 2, primers for three control sites (Amel, D5S818 and TH01) were also set. Primers for amplifying the full length of exons 1, 2a, 2b, 3, 4, 5, 6, and 8 of the SMN gene were also set, which can detect whether there are mutations related to exon length changes (such as Exon1 22insA mutation, Exon8 g.27706-27707 del AT mutation). There are also some relatively high incidence of pathogenic SNP sites and ARMS primers corresponding to the “2+0” related sites, which can be used with primers for amplifying exons to achieve the detection of target pathogenic SNPs.

The selection of pathogenic SNP sites is based on the relatively high incidence of pathogenic sites included in the OMIM database and the relatively high incidence of pathogenic sites reported in the literature (BMC Medical Genetics. 2012.13:86). The “2+0” related sites g.27134T>G and Exon8 g.27706-27707 del AT are from the reference (Human Mutation (2000) 15:228).

All SMA-related sites to be detected in the present disclosure include: 22insA, 683T>A, 400G>A, 689C>T, 830A>G, 835-1G>A, 863G>T, 5C>G, 305G>A, 815A>G, 821C>T, 785G>T, 399_402 del AGAG, g.27134T>G, and g.27706-27707 del AT of SMN1 gene.

In addition to primers, the PCR amplification system also contains the following components: DNA polymerase (2G Robust, KAPA Biosystems); UDPase enzyme; amplification buffer.

A total of 2802 samples (neonatal peripheral blood samples) were tested.

Each sample was amplified with two sets of primers in two reactions. The specific detection steps are the same as in Example 2.

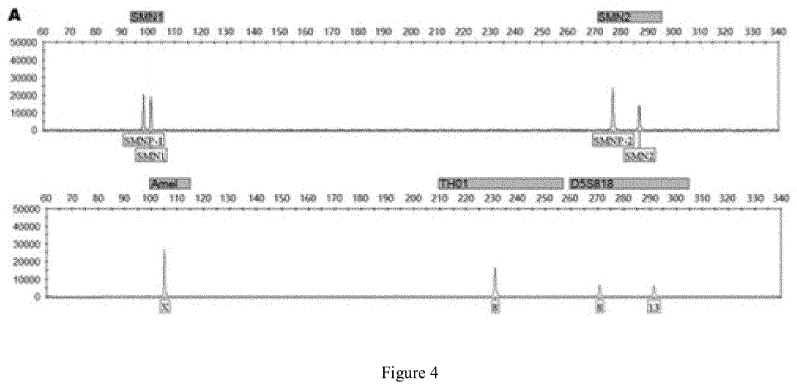

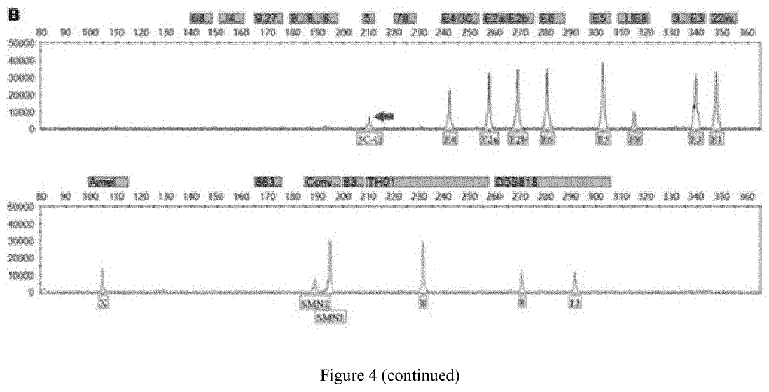

FIG. 4A shows the test result of the first reaction system of one of the samples. Comparing the peak areas of the corresponding products, it can be seen that the copy number of SMN1 is 2 and the copy number of SMN2 is 1.

FIG. 4B shows the test result of the second reaction system of the same sample. There is no conversion in this sample. Therefore, the copy number result obtained by the first system is an accurate result and does not need to be corrected. In particular, it is found that there is a product peak (shown by the arrow) at the corresponding position of 5C>G, indicating that the sample has 5C>G pathogenic mutation. Other control sites were normal, and there were no other target peaks of pathogenic mutations.

Among the 2802 samples, 4 cases of type II conversion were detected, and no type I conversion was detected.

After correction, 38 of the 2802 samples were detected as carriers, and the rest were normal people. The carrier frequency is 1.36%, which is basically the same as reported in the literature.

Among the 2802 samples, 1 case of pathogenic mutation was detected. That is, the 5C>G mutation was detected in the sample shown in FIG. 4.

In the 2802 samples, no “2+0” sample could be detected through two “2+0” related sites. It was speculated that the haplotype corresponding to the “2+0” related locus in the Jews does not exist or has a very low frequency in the Chinese population.

All carrier samples, conversion samples, pathogenic mutation samples, and 50 randomly selected normal samples were verified by MLPA or sequencing, and all results were consistent.

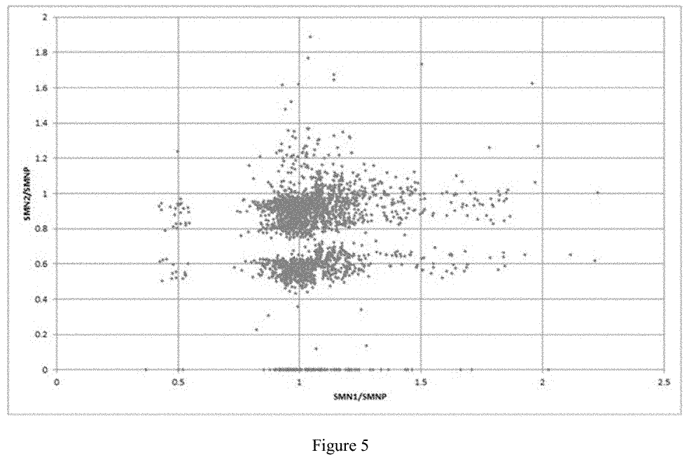

The scatter diagram is drawn based on the peak area ratio of 2802 samples detected by the first system, as shown in FIG. 5.

Wherein, the abscissa is the ratio of SMN1 and SMNP product peaks amplified simultaneously with SMN1-F and SMN1-R primers; the ordinate is the ratio of SMN2 and SMNP product peaks amplified simultaneously with SMN2-F and SMN2-R primers.

As can be seen from the figure, the data points corresponding to each sample are concentrated in several regions, and the boundaries between the regions are clear. Especially the points near 0.5 on the abscissa are clearly distinguished from other data points with larger abscissas. This shows that the test methods give excellent results in distinguishing 1 copy of SMN1 gene (SMA carriers) and 2 or more copies (normal people). Choosing SMNP as the control is the fundamental reason for the excellent quantitative and distinguishing ability of the test.

### Example 4 Detection of SMN1 and SMN2 Copy Numbers Based on NGS Platform

The technical solution provided by the present disclosure can be combined with other detection methods, such as NGS detection. A pair of primers was used to simultaneously amplify the sequence of SMN1/2 containing Exon7+6 site, and determine the copy number ratio of the two. At the same time, the sites that can satisfy the simultaneous amplification of SMN1, SMN2 and SMNP with the same primers are used as control sites to determine the total copy number of SMN1 and SMN2. Since the same primers and the same binding ability are used to simultaneously amplify and detect the corresponding regions of the three genes, the amplification test results can withstand various conditions and various interference factors, and the reads number of products corresponding to each gene can reflect the proportion of copy number of reaction template more accurately. Moreover, the total copy number of SMN1 and SMN2 may be determined effectively only by using a small number of sites, and there is no need to use a large number of control sites, which reduces system complexity and cost, and does not need to use more complex algorithms to modify the results.

1. PCR Amplification of Target Area in Samples

Primers for PCR amplification of the target region were designed to obtain the product of the target region. The target region includes SMN1, SMN2 and SMNP highly homologous regions, SMN1 and SMN2 differing base regions, SMN1/2 and some other gene exons and the area within a certain range of upstream and downstream thereof.

The primer sequence information is as follows:

| Primers | Sequence | Target gene |
|---|---|---|
| SMA-E1-F | AAGCGTGAGAAGTTACTACAAGC | SMN1/SMN2 |
| SMA-E1-R | CGCTAATAGGGAGACTGCACTG | |
| SMA-E2a-F | GTATGTGTGGATTAAGATGACTCT | SMN1/SMN2 |
| SMA-E2a-R | ATGTTATCAATTCCTTTCCAAATG | |
| SMA-E2b-F | GTCTGTGCACCACCCTGTAACAT | SMN1/SMN2 |
| SMA-E2b-R | ATAAGGACTAATGAGACATCCTTTG | |
| SMA-E3-F | GTAGTGGAAAGTTGGGGACAAA | SMN1/SMN2/ |
| | | SMNP |
| SMA-E3-R | GTATTTGCTCCTCTCTATTTCCATA | |
| SMA-E4-F | CAGTTTGATCCACCGAGGCATTA | SMN1/SMN2 |
| SMA-E4-R | ATTCTGGAAAACTTTCATAGAAGGT | |
| SMA-E5-F | GTTCCTATCATATTGAAATTGGTAAG | SMN1/SMN2 |
| SMA-E5-R | ACAATCCTCTATTCTGCTAATTATC | |
| SMA-E6-F | GTAAACAATATCTTTTTCTGTCTCC | SMN1/SMN2 |
| | AG | |
| SMA-E6-R | GTTGTCAGGAAAAGATGCTGAGTGAT | |
| SMA-E7-F | GACTATCAACTTAATTTCTGATCATA | SMN1/SMN2 |
| SMA-E7-R | GTTCATAATGCTGGCAGACTTAC | |
| SMA-17-F | AGTGAATCTTACTTTTGTAAA | SMN1/SMN2/ |
| SMA-17-R | AACCTTTTATCTAATAGTTTTGG | SMNP |
| SMA-17-F1 | CTACATCCCTACTAGAATTCTC | SMN1/SMN2/ |
| SMA-E8-R | GTCTGATCGTTTCTTTAGTGGTGT | SMNP |
| SMA-E8 1-F | GTTATAGAAGATAACTGGCCTCA | SMN1/SMN2/ |
| SMA-E8 1-R | CCTTCTCACAGCTCATAAAAT | SMNP |
| SMA-E8_2-F | GTTGCATACTTAAGCATTTAGGAA | SMN1/SMN2 |
| SMA-E8_2-R | AATGCTATGGTGGCATCCATATCA | |

The above primers cover all exons and part of the upstream and downstream sequences of SMN1/2.

There are 4 pairs of primers, SMA-E3-F/R, SMA-I7-F/R, SMA-I7-F1/SMA-E8-R, SMA-E8_1-F/R, which can simultaneously amplify the SMN1 and SMN2 sites and the control site SMNP with the same primers and the same binding ability.

1. Construction of Target Area Library for the Sample

The KAPA's Hyper Prep Kit was used to construct the library of the target region products amplified by the first step primers. According to the steps described in the instructions of the kit, perform blunting with addition of A, ligation, magnetic bead purification, library amplification and library purification, and the library construction of the sample target area was completed.

2. Sequencing

Illumina high-throughput sequencing platform was used for library sequencing, and NextSeq 500 System, Mid Output Flow Cell was used for PE150 sequencing.

3. Result Analysis

First, routine data analysis was performed, including:

Data processing software (NGSQCToolkit Version 2.3.3) was used to perform quality control on sequencing data (reads), and reads with low quality (lower than the quality requirements) (CutOffReadLen 80, CutOffQualScore 20) were removed.

The comparison software (BWA Version 0.7.15-r1140) was used to align the sequencing reads with the reference genome (hg19).

The sequencing depth was counted, using perl scripts to count the number of reads at specific locations.

The number of reads of the following 5 pairs of primers was analyzed: four pairs of primers that can simultaneously amplify SMN1, SMN2 and SMNP (SMA-E3-F/R, SMA-I7-F/R, SMA-I7-F1/SMA-E8-R, SMA-E8_1-F/R) and SMA-I7-F/R that amplifies exon 7. The reads of the amplified products of these primers provide direct information for determining the copy number of SMN1 and SMN2. All other loci detection results are related to pathogenic mutations, and have nothing to do with the determination of SMN1 and SMN2 copy numbers.

The SMA-I7-F/R amplified product contains the INS7+100 site. On the basis of distinguishing SMNP according to the sequence, SMN1 and SMN2 products can be distinguished according to the base of this position; SMA-I7-F1/SMA-E8-R amplified product contains the INS7+215 site. On the basis of distinguishing SMNP based on the sequence, the SMN1 and SMN2 products can be distinguished according to the base of this position; the SMA-E8_1-F/R amplified product contains Exon8+245 site, SMN1 and SMN2 products can be distinguished according to the base at this position on the basis of distinguishing SMNP according to sequence; SMA-E3-F/R does not contain a different site between SMN1 and SMN2, and it is impossible to distinguish between the two, only SMNP can be distinguished according to the sequence; SMA-E7-F/R amplified product contains Exon7+6 site, and SMN1 and SMN2 products can be distinguished based on the base of this position.

The statistics of the number of reads at these 5 sites of the 4 samples tested are as follows:

| | Reads of | Reads number | | | |
|---|---|---|---|---|---|
| Primer | corresponding gene | Sample 1 | Sample 2 | Sample 3 | Sample 4 |
| SMA-E3-F/R | SMN1/SMN2 | 3770 | 14875 | 16398 | 9663 |
| | SMNP | 3558 | 7030 | 8058 | 6192 |
| SMA-E7-F/R | SMN1 | 55 | 4127 | 10134 | 7170 |
| | SMN2 | 7590 | 12026 | 9992 | 3557 |
| | SMN1 | 49 | 3791 | 11216 | 7287 |
| SMA-I7-F/R | SMN2 | 6198 | 10137 | 11231 | 3715 |
| | SMNP | 6479 | 7603 | 12413 | 7456 |
| | SMN1 | 46 | 4623 | 3798 | 2384 |
| SMA-I7-F1/ | SMN2 | 8677 | 12328 | 3692 | 4408 |
| SMA-E8- R | SMNP | 7992 | 9327 | 3708 | 4534 |
| | SMN1 | 21 | 5153 | 20543 | 7765 |
| SMA-E8_1-F/R | SMN2 | 4613 | 14968 | 20571 | 15861 |
| | SMNP | 4879 | 11046 | 21360 | 14353 |

Exon7+6 site is the position really affects the function of SMN gene. The copy number detected by other sites such as INS7+100 is generally consistent with the copy number results of Exon7+6 site, but if the SMN1 and SMN2 genes are converted (such as sample 4), the measurement results will be biased.

The total copy number of SMN1 and SMN2 can be obtained from the sequencing results of the three sets of primers SMA-I7-F/R, SMA-I7-F1/SMA-E8-R, and SMA-E8_1-F/R. The ratio of SMN1 and SMN2 copy number can be obtained from the sequencing results of SMA-E7-F/R primer at Exon7+6 site. The exact copy number of SMN1 and SMN2 at Exon7+6 site can be obtained by combining the total copy number and copy number ratio of SMN1 and SMN2.

According to the number of reads of the 4 samples, the proportion of the corresponding number of reads is calculated, and the results are shown in the following table:

| Primer | | Sample 1 | Sample 2 | Sample 3 | Sample 4 |
|---|---|---|---|---|---|
| SMA-E3-F/R | (SMN1 + SMN2):SMNP | 1.06 | 2.12 | 2.03 | 1.49 |
| | (SMN1 + SMN2) copy number | 2 | 4 | 4 | 3 |
| SMA-E7-F/R | SMN1:SMN2 | 0.01 | 0.34 | 1.01 | 2.02 |
| | copy number ratio of SMN1 to | 0:N | 1:3 | 1:1 | 2:1 |
| | SMN2 | | | | |
| SMA-17-F/R | (SMN1 + SMN2):SMNP | 0.96 | 1.83 | 1.81 | 1.48 |
| | (SMN1 + SMN2) copy number | 2 | 4 | 4 | 3 |
| SMA-17-F1/ | (SMN1 + SMN2):SMNP | 1.09 | 1.82 | 2.02 | 1.50 |
| SMA-E8-R | (SMN1 + SMN2) copy number | 2 | 4 | 4 | 3 |
| SMA-E8_1-F/R | (SMN1 + SMN2):SMNP | 0.95 | 1.82 | 1.92 | 1.65 |
| | (SMN1 + SMN2) copy number | 2 | 4 | 4 | 3 |

In the four samples under test, the total copy number of SMN1 and SMN2 were the same at 4 sites where SMNP was amplified simultaneously. The total number of copies of the 4 samples are: 2, 4, 4, and 3.

According to the sequencing results of the SMA-E7-F/R primers, the copy number ratios of SMN1 and SMN2 of the 4 samples were 0:N, 1:3, 1:1, 2:1.

Combining the total copy number and copy number ratio of SMN1 and SMN2, it can be simply obtained that 4 copy number of SMN1 and SMN2 are 0/2, 1/3, 2/2, 2/1.

The above results are completely consistent with the MLPA results.

Due to the simultaneous amplification of SMNP sites, the detection of the total copy number of SMN1 and SMN2 is more accurate and simple, without the need to use additional control sites and complex algorithm corrections.

What is claimed is:

1. A method for detecting copy numbers of survival motor neuron SMN1 and/or SMN2 genes in a target genome, comprising
amplifying a target region in the SMN1 and/or SMN2 genes and SMNP gene in the target genome with a primer combination;
comparing amounts of amplified products of SMN1 and/or SMN2 genes by using SMNP amplified product as a reference; and
determining the copy numbers of SMN1 and/or SMN2 genes.

2. The method of claim 1, comprising
1) providing a sample containing target genomic DNA;
2) in the presence of the primer combination, using the genomic DNA of step 1) as a template and amplifying the target regions of SMN1 and/or SMN2 genes and SMNP gene recognized by the primer combination;
3) detecting products amplified by the primer combination, and using the SMNP amplified product as the reference to determine the copy numbers of SMN1 and/or SMN2 genes in the target genome; and
wherein,
the primer combination for determining SMN1 gene copy number is primer combination 1, which amplifies the target regions of SMN1 and SMNP genes but not SMN2 gene in the genome, and the SMN1 amplified product is distinguishable from the SMNP amplified product in the detection;
the primer combination for determining SMN2 gene copy number is primer combination 2, which amplifies the target region of SMN2 and SMNP genes but not SMN1 gene in the genome, and the SMN2 amplified product is distinguishable from the SMNP amplified product in the detection; and/or
the primer combination for determining total copy number of SMN1 and SMN2 genes is primer combination 3, which amplifies the target regions of SMN1, SMN2 and SMNP genes in the genome, and the SMNP amplified product is distinguishable from the amplified products of SMN1 and SMN2 in the detection.

3. The method of claim 2, wherein in step 3) the length and amount of the amplified products are detected, and wherein
for the primer combination 1, the lengths of the amplified products of SMN1 and SMNP are different;
for the primer combination 2, the lengths of the amplified products of SMN2 and SMNP are different; and/or
for the primer combination 3, the lengths of the amplified product of SMNP is different from the amplified products of SMN1 and SMN2.

4. The method of claim 2 or 3, wherein in step 3), the amplified products are detected by a method selected from the group consisting of fluorescence quantification, mass spectrometry and electrophoresis, such as capillary electrophoresis.

5. The method of claim 2, wherein in step 3), the sequences and amounts of the amplified products are detected.

6. The method of any one of claims 1 to 5, further comprising detecting gene conversion between the SMN1 and SMN2.

7. The method of any one of claims 2 to 6, wherein a first primer of the primer combination 1 is located in a first consensus sequence region of SMN1 and SMNP, and the sequence of the first primer is identical or complementary to at least a part of the first consensus sequence, for example, the first consensus sequence is SEQ ID NO: 1 (ATGAGAATTCTAGTAGGGATGTAG).

8. The method of claims 2 to 7, wherein a second primer of the primer combination 1 is located in a second consensus sequence region of SMN1 and SMNP, the corresponding sequence of SMN2 is not consistent with the second consensus sequence, and the sequence of the second primer is complementary or identical to at least a part of the second consensus sequence, for example, the second consensus sequence is SEQ ID NO: 2 (ATGTTAAAAAGTTGAAAGGTTAATGTAAAACA).

9. The method of any one of claims 2 to 8, wherein a third primer of the primer combination 2 is located in a third consensus sequence region of SMN2 and SMNP, the corresponding sequence of SMN1 is not consistent with the third consensus sequence, and the sequence of the third primer is complementary or identical to at least a part of the third consensus sequence, for example, the third consensus sequence is SEQ ID NO: 3 (ACTGGTTGGTTGTGTGGAA).

10. The method of any one of claims 2 to 9, wherein a fourth primer sequence of the primer combination 2 is located in a fourth consensus sequence region of SMN2 and SMNP, and the sequence of the fourth primer is complementary or identical to at least a part of the fourth consensus sequence, for example, the fourth consensus sequence is SEQ ID NO: 4 (GATCTGTCTGATCGTTTCTTTAGTGGTGTCATTTA) or SEQ ID NO: 5 (AATGAGGCCAGTTATCTTCTATAAC).

11. The method according to any one of claims 2 to 10, wherein the first primer and the second primer of the primer combination 1 comprise or consist of the sequences shown in SEQ ID NO: 6 and SEQ ID NO: 7 respectively; the third primer and the fourth primer of the primer combination 2 comprise or consist of the sequences shown in SEQ ID NO: 8 and SEQ ID NO: 9 respectively.

12. The method of any one of claims 6 to 11, wherein the primer combination further comprises primer combination 4 and/or primer combination 5 for detecting conversion between SMN1 and SMN2;
the primer combination 4 detects the conversion between INS7+100 site and Exon7+6 site;
the primer combination 5 detects the conversion between INS7+215 site and Exon7+6 site.

13. The method of claim 12, wherein the primer combination 4 comprises at least four primers, and the four primers comprise or consist of the sequences shown in SEQ ID NO: 10, SEQ ID NO: 11, SEQ ID NO: 12 and SEQ ID NO: 13, respectively; the primer combination 5 comprises at least four primers, and the four primers comprise or consist of the sequences shown in SEQ ID NO: 14, SEQ ID NO: 15, SEQ ID NO: 16 and SEQ ID NO: 17, respectively.

14. The method according to any one of claims 1 to 13, wherein at least one primer in the primer combination is modified or substituted with a modified base to replace a normal base, for example, the modification is selected from the group consisting of fluorescent group modification, phosphorylation modification, sulfur phosphorylation modification, locked nucleic acid modification and peptide nucleic acid modification.

15. The method of any one of claims 1 to 14, wherein the primer sequence in the primer combination has one or more nucleotide substitutions, additions, or deletions compared with the complementary sequence of the corresponding region on the template, while retaining its ability to initiate amplification reaction.

16. The method of any one of claims 1 to 15, wherein the amplification is performed by polymerase chain reaction (PCR).

17. The method of claim 16, wherein the PCR amplification is carried out in one or more reaction systems, such as 1, 2, 3, 4 or 5 reaction systems, and the primers of each of the PCR reaction systems are selected from the group consisting of primer combination 1, primer combination 2, primer combination 3, primer combination 4 and primer combination 5, and a combination thereof.

18. A method for diagnosing risk or severity of spinal muscular atrophy (SMA) in a subject or offspring thereof, comprising detecting the copy number of survival motor neuron genes SMN1 and/or SMN2 in the genome of the subject using the method according to any one of claims 1 to 17.

19. A kit for detecting copy number of SMN1 and/or SMN2 genes, comprising
primer combination 1 for determining the copy number of SMN1 gene, which amplifies target regions of SMN1 and SMNP genes but not SMN2 gene in the genome, and the SMN1 amplified product is distinguishable from the SMNP amplified product in the detection;
primer combination 2 for determining the copy number of SMN2 gene, which amplifies target regions of SMN2 and SMNP genes but not SMN1 gene in the genome, and the SMN2 amplified product is distinguishable from the SMNP amplified product in the detection; and/or
primer combination 3 for determining total copy number of SMN1 and SMN2 genes, which amplifies target regions of SMN1, SMN2 and SMNP genes in the genome, and the SMNP amplified product is distinguishable from the amplified products of SMN1 and SMN2 in the detection.

20. The kit of claim 19, wherein the primer combination 1 comprises a first primer and a second primer,
the first primer is located in a first consensus sequence region of SMN1 and SMNP genes, and the first primer sequence is identical or complementary to at least a part of the first consensus sequence, for example, the first consensus sequence is SEQ ID NO: 1 (ATGAGAATTCTAGTAGGGATGTAG), and
the second primer is located in a second consensus sequence region of SMN1 and SMNP genes, the corresponding sequence of the SMN2 gene is not consistent with the second consensus sequence, and the second primer sequence is complementary or identical to at least a part of the second consensus sequence, for example, the second consensus sequence is SEQ ID NO: 2 (ATGTTAAAAAGTTGAAAGGTTAATGTAAAACA).

21. The method of claim 19 or 20, wherein the primer combination 2 comprises a third primer and a fourth primer,
the third primer is located in a third consensus sequence region of SMN2 and SMNP genes, the corresponding sequence of the SMN1 gene is not consistent with the third consensus sequence, and the third primer sequence is complementary or identical to at least a part of the third consensus sequence, for example, the third consensus sequence is SEQ ID NO: 3 (ACTGGTTGGTTGTGTGGAA), and
the fourth primer is located in a fourth consensus sequence region of SMN2 and SMNP genes, and the fourth primer sequence is complementary or identical to at least a part of the fourth consensus sequence, for example, the fourth consensus sequence is SEQ ID NO: 4 (GATCTGTCTGATCGTTTCTTTAGTGGTGTCATTTA) or SEQ ID NO: 5 (AATGAGGCCAGTTATCTTCTATAAC).

22. The kit of any one of claims 19 to 21, wherein the first primer and the second primer comprise or consist of the sequences shown in SEQ ID NO: 6 and SEQ ID NO: 7 respectively; and the third primer and the fourth primer comprise or consist of the sequences shown in SEQ ID NO: 8 and SEQ ID NO: 9 respectively.

23. The kit according to any one of claims 19 to 22, further comprising primer combination 4 and/or primer combination 5 for detecting conversion between SMN1 and SMN2;
for example, the primer combination 4 detects the conversion between INS7+100 site and Exon7+6 site; and the primer combination 5 detects the conversion between INS7+215 site and Exon7+6 site.

24. The kit of claim 23, wherein the primer combination 4 comprises at least four primers, and the four primers comprise or consist of the sequences shown in SEQ ID NO: 10, SEQ ID NO: 11, SEQ ID NO: 12 and SEQ ID NO: 13, respectively; and the primer combination 5 comprises at least four primers, and the four primers comprise or consist of the sequences shown in SEQ ID NO: 14, SEQ ID NO: 15, SEQ ID NO: 16 and SEQ ID NO: 17, respectively.

25. The kit according to any one of claims 19 to 24, further comprising a primer combination 6 for detecting Amel gene related to spinal muscular atrophy and two STR sites D5S818 and TH01, and the primer sequences in the primer combination 6 are:

| Primer | Sequence | SEQ ID NO: |
|---|---|---|
| Amel-F | HEX-CCCTGGGCTCTGTAAAGAATAG | 18 |
| Amel-R | ATCAGAGCTTAAACTGGGAAGCTG | 19 |
| D5S818-F | HEX-CTCCCATCTGGATAGTGGACCT | 20 |
| D55818-R | ATAGCAAGTATGTGACAAGGGTG | 21 |
| Th01-F | HEX-AGGCTCTAGCAGCAGCTCATG | 22 |
| Th01-R | GAAAAGCTCCCGATTATCCAGCC | 23 |

26. The kit of any one of claims 19 to 25, wherein at least one primer is modified or substituted with a modified base to replace a normal base, for example, the modification is selected from the group consisting of fluorescent group modification, phosphorylation modification, phosphorothioate modification, locked nucleic acid modification and peptide nucleic acid modification.

27. The kit of any one of claims 19 to 26, wherein at least one primer sequence has one or more nucleotide replacements, additions, or deletions compared with the complementary sequence of the corresponding region on the template, while retaining its ability to initiate amplification reaction.

28. The kit of any one of claims 19 to 27, further comprising an instruction for use.
